(19) United States
(12) Patent Application Publication
(10) Pub. No.: US 2005/0271636 A1
(43) Pub. Date: Dec. 8, 2005

(54) Diagnostic and therapeutic uses for prox 1

(75) Inventors: Guillermo Oliver, Memphis, TN (US); Jeffrey Wigle, Manitoba (CA); Natasha Harvey, Walkerville (AU)

Correspondence Address: ST. JUDE CHILDREN'S RESEARCH HOSPITAL;OFFICE OF TECHNOLOGY LICENSING, 332 N. LAUDERDALE, MEMPHIS, TN, 38105, US

(73) Assignee: ST. JUDE CHILDREN'S RESEARCH HOSPITAL, INC., MEMPHIS, TN (US)

(21) Appl. No.: 10/523,292

(22) PCT Filed: Jul. 28, 2003
(86) PCT No.: PCT/US03/23584

§ 371 Date: Feb. 3, 2005

Related U.S. Application Data
(60) Provisional application No. 60/402,334, filed on Aug. 9, 2002.

(51) Int. Cl. A61K 48/00; G01N 33/574

(52) U.S. Cl. 424/93.21; 424/93.7; 435/7.23

(57) ABSTRACT

Methods and compositions based on the elucidation of the role of Prox1 in lymphatic tissue development in normal and tumor tissue are provided. Included are methods for determining the extent of lymphatic involvement in a tumor, methods for purifying endothelial precursor cells predisposed to develop into lymphatic tissue, and methods for promoting the development of lymphatic tissue. Pharmaceutical compositions and gene therapy vectors useful in the latter methods are provided.

### GOVERNMENT INTERESTS

This invention was made in part with U.S. Government support under National Institutes of Health grant nos EY12162 and GM58462 and was also supported by funds from the American Lebanese Syrian Associated Charities (ALSAC). The U.S. Government may have certain rights in this invention.

### FIELD OF THE INVENTION

The present invention relates generally to the development of lymphatic tissue and more particularly to methods for identifying lymphatic tissue and promoting the development and growth of lymphatic vessels.

### BACKGROUND OF THE INVENTION

The lymphatic system is a vascular network of thin-walled capillaries and larger vessels lined by a continuous layer of endothelial cells that drain lymph from the tissue spaces of most organs and return it to the venous system for recirculation. Although much information has been gained regarding the normal and pathological growth of the vascular system (Gale, N. W. and Yancopoulos, G. D. “Growth factors acting via endothelial cell-specific receptor tyrosine kinases: VEGFs, angiopoietins, and ephrins in vascular development,” Genes Dev 13: 1055-66 (1999).), the lack of specific lymphatic markers has made it difficult to elucidate the development of the lymphatic system. Consequently, the study of the formation of the lymphatic vasculature and its possible role in tumor metastasis has been neglected in the past, and the understanding of the precise manner by which the lymphatic system develops is still rudimentary.

Several reports have described the identification of novel lymphatic markers. See Karkkainen, M. J. et al, “Molecular regulation of lymphangiogenesis and targets for tissue oedema”, Trends Mol Med 7: 18-22 (2001); Wigle, J. T. et al, “Prox1 function is crucial for mouse lens-fiber elongation”, Nat Genet 21: 318-22 (1999); Jackson, D. G., et al. “Lyve-1, the lymphatic system and tumor lymphangiogenesis”, Trends Immunol 22:317-21 (2001); Banerji S., et al. “LYVE-1, a new homologue of the CD44 glycoprotein, is a lymph-specific receptor for hyaluronan”, J. Cell Biol. 144: 789-801(1999); and Nakano, H. and Gunn, M. D., “Gene duplications at the chemokine locus on mouse chromosome 4: multiple strain-specific haplotypes and the deletion of secondary lymphoid-organ chemokine and EBI-1 ligand chemokine genes in the plt mutation”, J Immunol 166: 361-9 (2001). Furthermore, other studies have provided evidence that both VEGF-C and VEGF-D, which are ligands for the vascular endothelial growth factor receptor 3 (VEGFR-3), can enhance tumor lymphangiogenesis and lymphatic metastasis. See Amioka, T. et al., “Vascular endothelial growth factor-C expression predicts lymph node metastasis of human gastric carcinomas invading the submucosa”, Eur. J. Cancer 10: 1413-1419 (July 2002); Makinen, T. et al., “Inhibition of lymphangiogenesis with resulting lymphedema in transgenic mice expressing soluble VEGF receptor-3”, Nat Med 7:199-205 (2001); Skobe, M. et al., “Induction of tumor lymphangiogenesis by VEGF-C promotes breast cancer metastasis” Nat Med 7: 192-8 (2001); Stacker, S. A. et al, “VEGF-D promotes the metastatic spread of tumor cells via the lymphatics”, Nat Med 7:186-91 (2001); Mandriota, S. J. et al., “Vascular endothelial growth factor-C-mediated lymphangiogenesis promotes tumour metastasis”, EMBO J. 20: 672-82 (2001); See also Padera, T. P. et al, “Lymphatic metastasis in the absence of functional intratumor lymphatics”, Science 296: 1883-1886 (June 2002). However, a detailed comparison of the expression patterns of these recently identified lymphatic markers during early stages of lymphatic development is not yet available.

Previous work validated the original proposal of the venous origin of the primary lymph sacs. Wigle, J. T. and Oliver, G., “Prox1 function is required for the development of the murine lymphatic system”, Cell 98:769-78 (1999); Sabin, F. R., “On the origin of the lymphatic system from the veins, and the development of the lymph hearts and thoracic duct in the pig”, AM J Anat 1:367-389 (1902). These results also indicated that the expression of the homeobox gene Prox1 in a restricted subpopulation of endothelial cells in the embryonic veins was required to promote lymphangiogenesis and that the initial localization and subsequent migration of the lymphatic endothelial cells from the cardinal vein were polarized (the endothelial cells appear to stream together along a defined pathway). This previous work also showed that in Prox1-null mice, budding and sprouting of lymphatic endothelial cells from the veins appears unaffected at E10.5. However, both processes are arrested prematurely at around E11.5-E12.0, and as a result of this arrest, Prox1-null mice are devoid of lymphatic vasculature (Wigle and Oliver 1999).

While previous research has shown that Prox1 is associated with normal lymphatic tissue, the exact role, if any, that it plays in lymphangiogenesis and in tumor tissue remains undetermined.

### SUMMARY OF THE INVENTION

In one aspect, the present invention provides a method for determining the extent of lymphatic involvement in a tumor based on the presence and/or distribution of Prox1 expression in or around the tumor. In this aspect, Prox1 expression is detected and measured at the mRNA or protein level using conventional techniques.

In another aspect, a method is provided for promoting the development of lymphatic vessels in a subject in need thereof, such as an individual suffering from lymphedema. This method comprises the provision of Prox1 protein to the subject's endothelial precursor cells. The inventors have identified a subpopulation of venous endothelial cells that behave as lymphatic precursors. Upon expression of Prox1 these precursors adopt a lymphatic vasculature phenotype. These endothelial precursor cells may be forced to express Prox1 protein and provided to an affected individual using available techniques, or Prox1 protein may be administered to the subject in the form of a gene therapy vector capable of expressing Prox1 in endothelial precursor cells.

In another aspect, a method for purifying those endothelial precursor cells that are predisposed to develop into lymphatic tissue is provided. These cells are purified based on their expression of Prox1 or LYVE1. Use of these purified cells to promote development of lymphatic tissue in subjects with lymphatic deficiencies is also taught.

### DETAILED DESCRIPTION OF THE INVENTION

The present invention is based in part upon the discovery of Prox1 as the best reliable lymphatic-specific marker and therefore a powerful tool to gauge the degree of lymphatic tissue development in or around tumors. The presence of intra or peritumoral lymphatic vessels may be used as a measure of the malignancy of a tumor.

Prox1 expression can be detected in tumor tissue samples using a variety of conventional methods. Suitable techniques particularly include the use of immunocytochemistry with a specific anti-Prox1 antibody, staining protein lysates with Prox1 antibody in Western Blot analyses, and methods that measure Prox1 mRNA levels. Although Prox1 is also expressed in other cell types (e.g lens fibers, hepatocytes), these other cell types can be easily distinguished from the endothelial cell type that gives rise to lymphatic tissue. For instance, the combination of the Prox1 antibody together with a panendothelial marker such as CD31 is an absolute confirmation of the lymphatic endothelial character of the stained tissue.

Reverse transcriptase-PCR (RT-PCR) is a preferred method for detecting Prox1 mRNA. Other RNA detection methods, e.g., in situ hybridization and northern blotting, PCR, real time PCR and ribonuclease protection assays can also be used. Because the sequence of the Prox1 gene and protein are publicly known, one can readily use conventional criteria to prepare suitable primers and probes for such methods. The nucleotide sequence of the human Prox1 gene can be found at genbank accession no. gi:21359845. The amino acid sequence of the human Prox1 protein can be found at genbank accession no. gi:21359846. The nucleotide sequence of the mouse Prox1 gene can be found at genbank accession no. gi:20834280. The amino acid sequence of the mouse Prox1 protein can be found at genbank accession no. gi:20834281.

### Prox1 Antibody

According to the present invention, Prox1 produced by a recombinant source, or through chemical synthesis, or isolated from a natural source; and derivatives or analogs thereof, including fusion proteins, may be used as an immunogen to generate antibodies that recognize Prox1. Such antibodies include but are not limited to polyclonal, monoclonal, chimeric including humanized chimeric, single chain, Fab fragments, and a Fab expression library. For example, a Prox1 polyclonal antibody has been raised in rabbit against a fusion protein made between the pGEX3 vector and the approximately 550 bp c-terminal BglII-EcoRI fragment of Prox1 containing all of the homeodomain and prox domain.

Anti-Prox1 antibodies may be cross reactive, that is, they may recognize Prox1 derived from a different source, e.g., an anti-mouse Prox1 antibody may recognize both human and mouse Prox1. Polyclonal antibodies have greater likelihood of cross reactivity. Alternatively, an antibody of the invention may be specific for a single form of Prox1, such as the mouse Prox1 or human Prox1.

Various procedures known in the art may be used for the production of polyclonal antibodies to Prox1 for example, or derivatives or analogs thereof. For the production of antibody, various host animals can be immunized by injection with Prox1 protein, or a derivative (e.g., or fusion protein) thereof, including but not limited to rabbits, mice, rats, sheep, goats, etc. In one embodiment, Prox1 can be conjugated to an immunogenic carrier, e.g., bovine serum albumin (BSA) or keyhole limpet hemocyanin (KLH). Various adjuvants may be used to increase the immunological response, depending on the host species, including but not limited to Freund's (complete and incomplete), mineral gels such as aluminum hydroxide, surface active substances such as lysolecithin, pluronic polyols, polyanions, peptides, oil emulsions, keyhole limpet hemocyanins, dinitrophenol, and potentially useful human adjuvants such as BCG (bacille Calmette-Guerin) and Corynebacterium parvum.

For preparation of monoclonal antibodies directed toward Prox1, or an analog or derivative thereof, any technique that provides for the production of antibody molecules by continuous cell lines in culture may be used. These include but are not limited to the hybridoma technique originally developed by [Kohler and Milstein Nature, 256:495-497 (1975)], as well as the trioma technique, the human B-cell hybridoma technique [Kozbor et al., Immunology Today, 4:72 (1983); Cote et al., Proc. Natl. Acad. Sci. USA, 80:2026-2030 (1983)], and the EBV-hybridoma technique to produce human monoclonal antibodies [Cole et al., in Monoclonal Antibodies and Cancer Therapy, Alan R. Liss, Inc., pp. 77-96 (1985)]. Monoclonal antibodies can be produced in germ-free animals utilizing recent technology [PCT/US90/02545]. Techniques developed for the production of “chimeric antibodies” [Morrison et al., J. Bacteriol., 159:870 (1984); Neuberger et al., Nature, 312:604-608 (1984); Takeda et al., Nature, 314:452454 (1985)] may also be used to make chimeric Prox1 antibodies.

Techniques described for the production of single chain antibodies [U.S. Pat. Nos. 5,476,786 and 5,132,405 to Huston; U.S. Pat. No. 4,946,778] can be adapted to produce Prox1 specific single chain antibodies. Techniques described for the construction of Fab expression libraries [Huse et al., Science, 246:1275-1281 (1989)] may be used to allow rapid and easy identification of monoclonal Fab fragments with the desired specificity for Prox1.

Antibody fragments which contain the idiotype of the antibody molecule can be generated by known techniques. For example, such fragments include but are not limited to: the F(ab□)₂ fragment which can be produced by pepsin digestion of the antibody molecule; the Fab□ fragments which can be generated by reducing the disulfide bridges of the F(ab□)₂ fragment, and the Fab fragments which can be generated by treating the antibody molecule with papain and a reducing agent.

In the production of antibodies, screening for the desired antibody can be accomplished by techniques known in the art, e.g., radioimmunoassay, ELISA (enzyme-linked immunosorbant assay), “sandwich” immunoassays, immunoradiometric assays, gel diffusion precipitin reactions, immunodiffusion assays, in situ immunoassays (using colloidal gold, enzyme or radioisotope labels, for example), Western blots, precipitation reactions, agglutination assays (e.g., gel agglutination assays, hemagglutination assays), complement fixation assays, immunofluorescence assays, protein A assays, flow cytometry, and immunoelectrophoresis assays, etc. In one embodiment, antibody binding is detected by detecting a label on the primary antibody. In another embodiment, the primary antibody is detected by detecting binding of a secondary antibody or reagent to the primary antibody. In a further embodiment, the secondary antibody is labeled. Many means are known in the art for detecting binding in an immunoassay and are within the scope of the present invention. For example, to select antibodies which recognize a specific epitope of Prox1, one may assay generated hybridomas for a product which binds to the Prox1 fragment containing such epitope.

Prox1 antibodies used in the methods of the present invention can be labeled using conventional technology. Suitable labels include enzymes, fluorophores (e.g., fluorescein isothiocyanate (FITC), phycoerythrin (PE), Texas red (TR), rhodamine, free or chelated lanthanide series salts, especially Eu³⁺, to name a few fluorophores), chromophores, radioisotopes, chelating agents, dyes, colloidal gold, latex particles, ligands (e.g., biotin), and chemiluminescent agents. In the instance where a radioactive label, such as the isotopes ³H, ¹⁴C, ³²P, ³⁵S, ¹²⁵I, and ¹³¹I, are used, known currently available counting procedures may be utilized. In the instance where the label is an enzyme, detection may be accomplished by any of the presently utilized colorimetric, spectrophotometric, fluorospectrophotometric, amperometric or gasometric techniques known in the art.

Direct labels are one example of labels which can be used with Prox1 antibodies. A direct label has been defined as an entity, which in its natural state, is readily visible, either to the naked eye, or with the aid of an optical filter and/or applied stimulation, e.g. ultraviolet light to promote fluorescence. Among examples of colored labels, which can be used according to the present invention, include metallic sol particles, for example, gold sol particles such as those described by Leuvering (U.S. Pat. No. 4,313,734); dye sole particles such as described by Gribnau et al. U.S. Pat. No. 4,373,932) and May et al. (WO 88/08534); dyed latex such as described by May, supra, Snyder (EP-A 0 280 559 and 0 281 327); or dyes encapsulated in liposomes as described by Campbell et al. (U.S. Pat. No. 4,703,017). Other direct labels include a radionucleotide, a fluorescent moiety or a luminescent moiety.

In addition to these direct labeling devices, indirect labels comprising enzymes can also be used according to the present invention. Various types of enzyme linked immunoassays are well known in the art, for example, alkaline phosphatase and horseradish peroxidase, lysozyme, glucose-6-phosphate dehydrogenase, lactate dehydrogenase, urease, these and others have been discussed in detail by Eva Engvall in Enzyme Immunoassay ELISA and EMIT in Methods in Enzymology, 70:419439 (1980) and in U.S. Pat. No. 4,857,453.

In addition, an antibody can be modified to contain a marker protein such as green fluorescent protein as described in U.S. Pat. No. 5,625,048 filed Apr. 29, 1997, WO 97/26333, published Jul. 24, 1997 and WO 99/64592 all of which are hereby incorporated by reference in their entireties. Other labels for use in the invention include magnetic beads or magnetic resonance imaging labels.

A phosphorylation site can also be created on a Prox1 antibody for labeling with ³²P, e.g., as described in European Patent No. 0372707 by Sidney Pestka, or U.S. Pat. No. 5,459,240 issued Oct. 17, 1995 to Foxwell et al.

Prox1 antibodies also can be labeled by metabolic labeling. Metabolic labeling occurs during in vitro incubation of the cells that express the protein in the presence of culture medium supplemented with a metabolic label, such as [³⁵S]-methionine or [³²P]-orthophosphate. In addition to metabolic (or biosynthetic) labeling with [³⁵S]-methionine, the invention further contemplates labeling with [¹⁴C]-amino acids and [³H]-amino acids (with the tritium substituted at non-labile positions).

The present invention is also based upon the elucidation of the critical role Prox1 plays in the development of lymphatic tissue. The present invention teaches that Prox1 may be used to promote the development of lymphatic tissue from endothelial precursor cells. This may be accomplished by providing Prox1 in the form of a DNA vector designed to express Prox1 in vascular endothelial precursor cells. The presence of Prox1 in such cells promotes their development into lymphatic tissue.

### Gene Therapy Vectors

The Prox1 gene can be introduced into endothelial precursor cells to develop gene therapy for conditions that result in lymphatic deficiency. Such therapy would be expected to increase lymphatic tissue development from vascular endothelial precursor cells. Conversely, introduction of antisense constructs into tumor cells would reduce the levels of active Prox1 and would be predicted to decrease lymphatic involvement in the tumor.

Vectors designed to express Prox1 in endothelial precursor cells may be constructed using standard components and techniques. Promoter elements know to drive specific expression in blood vascular endothelial cells (e.g. Tie2) may be used to ectopically express Prox1 in those cell types.

In one embodiment, a gene encoding Prox1 is introduced in vivo in a viral vector. Such vectors include an attenuated or defective DNA virus, such as but not limited to herpes simplex virus (HSV), papillomavirus, Epstein Barr virus (EBV), adenovirus, adeno-associated virus (AAV), and the like. Defective viruses, which entirely or almost entirely lack viral genes, are preferred. Defective virus is not infective after introduction into a cell. Use of defective viral vectors allows for administration to cells in a specific, localized area, without concern that the vector can infect other cells. Thus, tissue containing endothelial precursor cells can be specifically targeted. Examples of particular vectors include, but are not limited to, a defective herpes virus 1 (HSV1) vector described by Kaplitt et al., Molec. Cell. Neurosci. 2:320-330 (1991)), an attenuated adenovirus vector, such as the vector described by Stratford-Perricaudet et al., J. Clin. Invest. 90:626-630(1992), and a defective adeno-associted virus vector as described by Samulski et al., J. Virol. 61:3096-3101 (1987) and Samulski et al., J. Virol. 63:3822-3828 (1989).

In another embodiment the gene can be introduced in a retroviral vector, e.g., as described in Anderson et al., U.S. Pat. No. 5,399,346; Mann et al., Cell 33:153 (1983); Temin et al., U.S. Pat. No. 4,650,764; Temin et al., U.S. Pat. No. 4,980,289; Markowitz et al., J. Virol. 62:1120 (1988); Temin et al., U.S. Pat. No. 5,124,263; International Patent Publication No. WO 95/07358, published Mar. 16, 1995, by Dougherty et al.; and Kuo et al., Blood 82:845 (1993).

Alternatively, the vector can be introduced in vivo by lipofection. There has been increasing use of liposomes for encapsulation and transfection of nucleic acids in vitro. Synthetic cationic lipids designed to limit the difficulties and dangers encountered with liposome mediated transfection can be used to prepare liposomes for in vivo transfection of a gene encoding a marker (Felgner, et. al., Proc. Natl. Acad. Sci. U.S.A. 84:7413-7417 (1987); see Mackey, et al., Proc. Natl. Acad. Sci. U.S.A. 85:8027-8031 (1988)). The use of cationic lipids may promote encapsulation of negatively charged nucleic acids, and also promote fusion with negatively charged cell membranes (Felgner and Ringold, Science 337:387-388 (1989)). The use of lipofection to introduce exogenous genes into the specific organs in vivo has certain practical advantages. Molecular targeting of liposomes to specific cells represents one area of benefit. It is clear that directing transfection to particular cell types would be particularly advantageous in a tissue with cellular heterogeneity, such as pancreases liver, kidney, and the brain. Lipids may be chemically coupled to other molecules for the purpose of targeting (see Mackey, et. al., 1988, supra). Targeted peptides, e.g., hormones or neurotransmitters, and proteins such as antibodies, or non-peptide molecules could be coupled to liposomes chemically.

It is also possible to introduce the vector in vivo as a naked DNA plasmid. Naked DNA vectors for gene therapy can be introduced into the desired host cells by methods known in the art, e.g., transfection, electroporation, microinjection, transduction, cell fusion, DEAE dextran, calcium phosphate precipitation, use of a gene gun, or use of a DNA vector transporter (see, e.g., Wu et al., J. Biol. Chem. 267:963-967 (1992); Wu and Wu, J. Biol. Chem. 263:14621-14624 (1988); Hartmut et al., Canadian Patent Application No. 2,012,311 filed Mar. 15, 1990).

The methods taught herein for promoting the development of lymphatic tissue are particularly useful when applied to subjects suffering from any lymphatic disorder, for example lymphoedema resulting after radiation treatment in breast cancer patients.

### Purifying Prox1 Expressing Cells

In another aspect, a method of purifying endothelial precursor cells having the potential to develop into lymphatic tissue is provided. This method is based on the selective expression of Prox1 and lymphatic endothelial hyaluronan (HA) receptor (LYVE-1) in this subpopulation of endothelial precursor cells. Embryonic veins may be used as the starting material for this isolation. Additionally, isolated blood vascular endothelial cells (purified by cell sorting) could also be infected with virus expressing Prox1 cDNA which will induce the lymphatic differentiation program in these cells.

According to this method, cells expressing Prox1 and/or LYVE-1 are purified from the starting material. This may be accomplished by cell sorting using antibodies that recognize LYVE-1 and/or Prox1 and may also include antibodies that recognize the pan-endothelial marker CD31. These cells represent endothelial precursor cells having the potential to develop into lymphatic tissue.

Antibodies that recognize CD31 are available from Pharmingen, San Diego, Calif. or they may be generated using the CD31 protein as an immunogen. The CD31 protein can be produced recombinantly using the CD31 coding sequence (genbank accession number gi:585658). Antibodies that recognize LYVE-1 may be generated using the LYVE-1 protein as an immunogen. The LYVE-1 protein can be produced recombinantly using the LYVE-1 coding sequence available from genbank at accession no. gi:13640029].

Endothelial precursor cells having the potential to develop into lymphatic tissue purified according to this method may be administered to a compatible subject to promote lymphatic tissue development. Such administration may be particularly useful for subjects suffering from lymphatic disorders as listed above and represents an alternative approach for treating these disorders.

The present invention may be better understood by reference to the following non-limiting examples. These examples are presented in order to more fully illustrate the invention through the description of particular embodiments. These examples should in no way be construed as limiting the scope of the invention.

### EXAMPLES

### Example 1

### An Essential Role for Prox1 in the Induction of the Lymphatic Endothelial Cell Phenotype

Summary

The process of angiogenesis has been well documented, but little is known about the biology of lymphatic endothelial cells and the molecular mechanisms that control lymphangiogenesis. The homeobox gene Prox1 is expressed in a subpopulation of endothelial cells that, after budding from veins, gives rise to the mammalian lymphatic system. In Prox1^{-/-} embryos, this budding becomes arrested at around embryonic day (E) 11.5; the results of this arrest are embryos without lymphatic vasculature. Unlike the endothelial cells that bud off in E11.5 wild-type embryos, those of Prox1-null embryos did not co-express any lymphatic markers such as VEGFR-3, LYVE-1, or SLC. Instead, the mutant cells appeared to have a blood vascular phenotype, as determined by their expression of laminin and CD34. These results indicate that Prox1 activity is required not only for maintenance of the budding of the venous endothelial cells but also for differentiation toward the lymphatic phenotype. On the basis of our findings, we contemplate that a blood vascular phenotype is the default fate of budding embryonic venous endothelial cells; upon expression of Prox1, these budding cells adopt a lymphatic vasculature phenotype. In addition, we have also determined that Prox1, VEGFR-3, LYVE-1, and SLC are similarly expressed in lymphatic endothelial cells of normal adult and tumor tissues.

Results

Lymphatic Markers During Early Embryonic Development

To determine the mechanisms by which Prox1 regulates the budding and sprouting of lymphatic endothelial cells, we initially compared the expression of Prox1 during early murine embryonic development with that of other lymphatic markers.

The first indication that lymphangiogenesis has begun is the specific expression of Prox1 in a restricted subpopulation of endothelial cells in the anterior cardinal vein at E9.5 (Wigle and Oliver 1999). In wild-type embryos at E10.5, the restricted localization of Prox1 in the veins was still evident, and the first lymphatic endothelial cells had started to bud off in a polarized (not random) manner. In previous studies it has been shown that as embryonic development proceeds, expression of the gene encoding vascular endothelial growth factor receptor-3 (VEGFR-3) becomes largely restricted to the lymphatic vessels; with lower levels of expression remaining in blood vessels (Kaipainen, A. et al, “Expression of the fms-like tyrosine kinase 4 gene becomes restricted to lymphatic endothelium during development”, Proc Nat Acad Sci USA 92: 3566-70 (1995); Wigle and Oliver 1999). In this study we also detected high levels of VEGFR-3 expression in the budding Prox1-positive endothelial cells at E10.5; however, VEGFR-3 expression, although less pronounced, was still detected in the arteries and veins at that time. At E10.5, the expression pattern of Prox1 resembles that of lymphatic endothelial hyaluronan (HA) receptor (LYVE-1) in the budding endothelial cells. LYVE-1 is a member of the Link protein superfamily that was recently identified as a cell surface protein of lymphatic endothelial cells (Banerji et al. 1999; Prevo, R. et al, “Mouse LYVE-1 is an endocytic receptor for hyaluronan in lymphatic endothelium”, J Biol Chem 276:19420-30 (2001); Jackson et al. 2001). The only difference between the expression pattern of Prox1 and LYVE-1 at this stage was that LYVE-1 was uniformly expressed in the endothelial cells of the cardinal vein, whereas Prox1 expression in the vein was only detected in a restricted subpopulation of endothelial cells.

Interactions between LYVE-1 and the extracellular matrix glycosaminoglycan HA might regulate leukocyte migration through the lymphatic vasculature (Jackson et al. 2001). However, chemokines such as secondary lymphoid chemokine (SLC or CCL21), which are released by the lymphatic endothelium (Zlotnik, A. and Yoshie, O., “Chemokines: a new classification syste and their role in immunity”, Immunity 12:121-7 (2000); Gunn, M. D. et al, “A chemokine expressed in lymphoid high endothelial venules promotes the adhesion and chemotaxis of naïve T lymphocytes”, Proc Nat Acad Sci USA 95:258-63 (1998); Gunn, M. D. et al, “Mice lacking expression of secondary lymphoid organ chemokine have defects in lymphocyte homing and dendritic cell localization”, J Exp Med 189:451-60 (1999), regulate the attraction of leukocytes toward the lymphatic vessels. At E10.5, budding lymphatic endothelial cells had not yet begun to express SLC, a finding that supports the hypothesis that these early budding endothelial cells (Prox1, LYVE-1, and VEGFR-3-positive) are in the early stages of the lymphangiogenic pathway prior to leukocyte intravasation.

The number and distribution of lymphatic endothelial cells that had budded from the veins was greatly increased between E10.5 and E12.5. At E12.5, the number of Prox1-and LYVE-1-positive cells adjacent to the cardinal vein had clearly increased, but Prox1 and LYVE-1 expression was no longer detected in endothelial cells in the cardinal vein. In the lymphatic endothelial cells, levels of VEGFR-3 remained high whereas its expression in vascular endothelial cells had substantially diminished. SLC expression was initially detected in only a subset of the budding lymphatic endothelial cells at E11.5. At E12.5, the pattern of SLC expression, while more patchy in appearance, was almost identical to that of the other lymphatic markers. Maintenance of high levels of VEGFR-3 expression in the lymphatic endothelial cells, together with a reduction of its expression level in the vascular endothelial cells, as well as the beginning of expression of SLC in the Prox1- and LYVE-1-positive endothelial cells that had already budded off from the veins, is an indication that these cells are now committed (biased) to the lymphatic pathway (lymphatic precursors).

As previously shown (Wigle and Oliver 1999) in Prox1 heterozygous embryos at E14.5, the lymphatic vasculature has spread throughout the embryo. Prox1-positive endothelial cells co-expressed high levels of VEGFR-3, whereas Prox1-negative cells expressed low levels of this receptor, indicating that these cells are blood vascular endothelia. The patterns of expression of LYVE-1 and SLC in adjacent sections were also similar to that of Prox1; however, LYVE-1 was also expressed in scattered non-lymphatic endothelial cells that corresponded to macrophages. In contrast to the well-developed lymphatic vasculature found in heterozygous E14.5 embryos, no lymphatic vasculature was present in a similar section from a Prox1-nullizygous littermate (Wigle and Oliver 1999). As demonstrated previously (Wigle and Oliver 1999), the development of the blood vasculature had progressed normally as indicated by the abundant expression of the platelet endothelial cell adhesion molecule (PECAM). The lymphatic endothelial cells that expressed high levels of VEGFR-3 were no longer present, and only cells that expressed low levels of VEGFR-3 were still detected in the blood vasculature. The capillary-like staining observed for LYVE-1 in the E14.5 heterozygous embryos was absent in Prox1-null littermates and the only remaining staining corresponded to scattered macrophages. No endothelial-specific SLC expression was detected in the nullizgyous embryos at this timepoint.

Lymphatic Markers in Normal Adult and Tumor Tissues

In an effort to determine whether Prox1 might serve as a reliable marker of adult lymphatic vasculature, and whether the vasculature of different tumors express lymphatic markers in patterns that differ from those of normal adult lymphatic vasculature, we extended the comparison of the markers we used to characterize the embryonic lymphatic vasculature in the embryo to two different tumor types that were surrounded by normal adult tissue.

Recent results have provided experimental evidence indicating that tumors can activate lymphangiogenesis, and that some vascular/lymphatic endothelial growth factors (VEGF-C and VEGF-D) can enhance lymphatic metastasis (Makinen et al. 2001; Skobe et al. 2001; Stacker et al. 2001; Mandriota et al. 2001). It has also been shown that angiosarcomas express markers for blood and lymphatic capillaries (Breiteneder-Geleff, S. et al., “Angiosarcomas express mixed endothelial phenotypes of blood and lymphatic capillaries: podoplanin as a specific marker for lymphatic endothelium”, Am J Pathol 154: 385-94 (1999).

In a xenografted A431 human squamous cell carcinoma, Prox1-positive lymphatic vasculature was detected in the normal adult dermis adjacent to the tumor and occasionally, within the tumor itself. In adjacent sections, the Prox1-positive cells also co-expressed VEGFR-3, and weaker VEGFR-3 expression was detected in intratumoral blood vessels. All Prox1-positive vessels, including those proximal and those distal from the tumor, also expressed SLC and LYVE-1. Intratumoral LYVE-1 and Prox1 expression was also observed. Similar results were also in orthotopic carcinomas induced by a chemical carcinogenesis regimen.

To determine whether Prox1 is a general marker of adult and tumor-associated lymphatic vasculature, we analyzed Prox1 expression in a spontaneous highly angiogenic lymphoma that developed in the leg of an Ink4d (p19^{ARF}) mutant mouse and that was also surrounded by abundant normal tissue. Similar to the squamous cell carcinoma, Prox1, VEGFR-3, LYVE-1 and SLC expression were detected in lymphatic vessels adjacent to the tumor.

Phenotypic Characterization of Endothelial Cells of Prox1 Nullizygous Embryos

After we validated Prox1, VEGFR-3, LYVE-1, and SLC as suitable markers of the embryonic and adult lymphatic vasculature, we undertook the precise phenotypic characterization of the Prox1 null embryos at E11.5. At E10.5, lymphatic endothelial cell precursors bud off from the veins in normal numbers in Prox1 nullizygous embryos. However, starting at around E11.5 fewer than normal budding Prox1-positive (β-galactosidase-positive, β-gal) endothelial cells were detected in Prox1 nullizygous embryos (Wigle and Oliver 1999), and this budding was no longer polarized, but instead, the endothelial cells followed a random migratory path. As expected, the β-gal-positive endothelial cells in the heterozygous embryos also exhibited high levels of VEGFR-3 expression. Surprisingly, only weak VEGFR-3 expression was observed in the endothelial cells of the Prox1-null littermates, indicating that those cells most likely corresponded to blood vasculature cells. In heterozygous embryos at this stage, LYVE-1 is weakly expressed in the cardinal vein and strongly expressed in the budding β-gal-positive endothelial cells. The expression of LYVE-1 overlapped with that of β-gal in the cardinal vein but not with that in the budding β-gal-positive endothelial cells of Prox1-nullizygous littermates. Some heterozygous endothelial cells started to express SLC at this stage; however, none of the β-gal-positive endothelial cells of the nullizygous embryos expressed detectable levels of this chemokine.

Expression of blood vascular markers in endothelial cells of Prox1 homozygous embryos Unlike the lymphatic system, the blood vessels have a distinct continuous basal membrane that contains laminin (Ezaki et al, “A new approach for identification of rat lymphatic capillaries using a monoclonal antibody”, Arch Histol Cytol 53 (Suppl): 77-86 (1990). In addition, and in contrast to lymphatic endothelial cells, blood endothelial cells express high levels of the surface glycoprotein CD34 (Paal et al., “A clinicopathologic and immunohistochemical study of ten pancreatic lymphangiomas and a review of the literature”, Cancer 82:2150-8 (1998); Breiteneder-Geleff et al., 1999). Therefore, we used both of these markers to help identify the β-gal-positive endothelial cells present in Prox1-null embryos at E11.5. We now confirmed by double-labeling immunohistochemistry that in contrast to the wild-type embryo in which the budding 13-gal-positive endothelial cells coexpressed LYVE-1, in the mutant littermate they did not. In the heterozygous embryos, 13-gal-positive endothelial cells budding from the cardinal vein did not co-express laminin and only expressed very low to undetectable levels of CD34. In the mutant littermates, budding LacZ-expressing cells expressed high levels of laminin and CD34. These results indicated that in Prox1-null embryos, the budding endothelial cells which are still detected at E11.5-12.0 have adopted a blood vascular, instead of the wild-type lymphatic phenotype. In addition, we have determined that already at E10.5, the 13-gal-positive endothelial cells budding from the cardinal vein of the mutant embryos do not co-express LYVE-1. However, the differences in the levels of expression of laminin and CD34 between the wild-type and the mutant littermates are not yet as obvious as at E11.5.

Discussion

The lack of specific markers has hampered the understanding of the mechanisms controlling the development of the lymphatic vascular system. We have previously shown that Prox1 plays a key role in lymphangiogenesis (Wigle and Oliver 1999). We found that Prox1 activity is not required to initiate budding of endothelial cells from the cardinal vein, but instead to maintain the budding and sprouting of a restricted subpopulation of endothelial cells that give rise to the lymphatic vasculature (Wigle and Oliver 1999). By comparing the expression of lymphatic-and blood vascular-specific markers in Prox1 heterozygous and nullizygous embryos and in normal adult tissues and tumors, we have further elucidated the role of Prox1 in the development and maintenance of the lymphatic system.

To determine the phenotypic properties of Prox1-positive cells in heterozygous and nullizygous Prox1 embryos, we investigated the expression of three other available lymphatic markers (VEGFR-3, LYVE-1, SLC) and two blood vascular markers (laminin, CD34). Functional inactivation of VEGFR-3 in mice disrupts the development of the cardiovascular system (Dumont, D. J. et al. “Cardiovascular failure in mouse embryos deficient in VEGF receptor-3”, Science 282: 946-9 (1998)). VEGFR-3 is expressed in the endothelial cells of some fenestrated blood vessels (Partanen, T. A. et al. “VEGF-C and VEGF-D expression in neuroendocrine cells and their receptor, VEGFR-3, in fenestrated blood vessels in human tissues”, FASEB J 14: 2087-96 (2000) and in angiogenic blood vessels in some tumors (Valtola, R. et al. “VEGFR-3 and it s ligand VEGF-C are associated with angiogeneis in breast cancer”, Am J Pathol 154: 1381-90 (1999). However, during later embryonic development, VEGFR-3 expression becomes largely restricted to the lymphatic vessels (Kaipanen et al. 1995; Wigle and Oliver 1999). The key role that this marker plays in the lymphatic system was demonstrated by the identification of mutations in VEGFR-3 in several cases of congenital lymphoedema (Karkkainen, M. J. et al., “Missense mutations interfere with VEGFR-3 signalling in primary lymphoedema”, Nat Genet 25: 153-9 (2000).

LYVE-1, a member of the Link protein superfamily, was recently identified as a lymph-specific receptor for the extracellular matrix glycosaminoglycan HA (Banerji et al. 1999). HA is thought to provide a hydrated environment that facilitates cell transformation and migration during development (Jackson et al. 2001). LYVE-1 may also participate in the uptake or transport of HA across the lymphatic wall (Prevo et al. 2001; Jackson et al. 2001). Immunohistochemical analyses have demonstrated LYVE-1 expression on the surface of endothelial cells of lymphatic vessels (Prevo et al. 2001; Jackson et al. 2001).

SLC (or CCL21) is released by the lymphatic endothelium (Zlotnick and Yoshie 2000; Gunn et al. 1998, 1999). The migration of leukocytes toward cells comprising the lymphatic vasculature is regulated, at least in part, by this chemokine. SLC is uniformly expressed in adult lymphatic endothelium (Gunn et al. 1998, 1999), and is expressed in embryonic lymphatics as early as E11.5.

In the present study, we found that β-gal-positive endothelial cells that start to bud from the veins of Prox1-null embryos, but are arrested at E11.5-12.0, do not undergo lymphatic differentiation. These cells do not express any of the lymphatic markers except VEGFR-3 (at low levels), but they do express high levels of markers such as laminin and CD34, a finding that suggests that these cells have adopted a blood vascular phenotype.

In addition to the arrest of endothelial cell budding and migration observed in Prox1-null embryos at around E11.5, the polarity (directionality) of the budding was also defective. This finding suggests that Prox1 function is required for normal maintenance of some as yet unidentified signaling mechanism that is involved in guiding the budding and migration of the lymphatic endothelial cells. This molecule may be located in the surrounding tissue on one side of the cardinal vein, and its activity is dependent on Prox1 function, in a yet undetermined cell autonomous or non-cell autonomous manner.

Our findings suggest that Prox1 activity is required not only to maintain budding and sprouting of a subpopulation of venous endothelial cells that will give rise to the lymphatic vasculature but also to determine the final fate of those budding endothelial cells. On the basis of our results, we have developed a working model of early lymphatic vascular development. After the initial formation of the vascular system, the expression of LYVE-1 and Prox1 in endothelial cells in the cardinal veins at approximately E9.5 to E10.0 is likely one of the first indications that lymphangiogenesis has been initiated. All endothelial cells in the veins are probably initially bipotent, and upon the expression of at least Prox1 in a restricted subpopulation of venous endothelial cells (on only one side of the cardinal vein), those cells become committed (biased) to initiate the lymphatic differentiation program. As development proceeds, this subpopulation of LYVE-1 and Prox1-positive endothelial cells starts to bud from the veins in an initially Prox1-independent manner. However, maintenance of the budding and migration requires Prox1 activity. Normally, as the cells bud in a polarized manner, they start to express additional lymphatic endothelial markers. At this stage, SLC expression is first detected, and VEGFR-3 expression is maintained at high levels in budding lymphatic endothelial cells, but its expression becomes weaker in blood vascular endothelial cells. The expression of these four lymphatic markers may indicate that this process becomes irreversibly specified toward the lymphatic pathway. On the basis of our results, we contemplate that this step is also dependent on Prox1 activity and on a feedback signaling mechanism required for the maintenance and polarized budding of these lymphatic endothelial cells. Endothelial cell budding and migration is arrested because of the lack of Prox1 function, and random budding occurs because of a failure in the feedback-loop signaling mechanism. As a result, neither lymphatic bias nor lymphatic specification is accomplished. Therefore, Prox1 activity in a restricted subpopulation of endothelial cells in the embryonic veins is required not only to promote lymphangiogenesis but also to determine the lymphatic fate by the initiation of the lymphatic differentiation program of those budding venous endothelial cells. In the future, the identification of the molecules and the mechanisms involved in these developmental decisions will provide important information for our understanding of lymphangiogenesis during development and in diseases such as cancer.

Materials and Methods

Animals: Prox1 heterozygous and Ink4d nullizygous mice were generated as previously described (Wigle et al. 1999; Kamijo, T. et al. “Tumor suppression at the mouse locus mediated by the alternative reading frame product p19^{ARF}”, Cell 91: 649-59 (1997); these methods followed NIH-approved institutional animal care guidelines.

Immunohistochemistry: Embryos were dissected and fixed in 4% paraformaldehyde by constant shaking at 4° C. for a period of 1 h to overnight. The embryos were cryoprotected in 30% sucrose dissolved in phosphate-buffered saline (PBS), embedded in tissue-freezing medium (Triangle Biomedical Sciences, Durham, N.C.), and cut into 10-μm sections on a cryostat. Sections for immunohistochemical analyses were treated with Avidin/Biotin Blocking Kit (Vector Laboratories, Burlingame, Calif.) before the primary antisera were added.

The following primary antibodies were used in this study; rabbit anti-Prox1, rabbit anti-mouse LYVE-1 (Prevo et al. 1999), rat anti-mouse LYVE-1 (polyclonal serum generated against mouse LYVE-1 Fc); goat anti-VEGFR-3/Flt4 and goat anti-C6-kine/Secondary Lymphoid Chemokine (R&D Systems, Minneapolis, Minn.), rat anti-PECAM-1/CD31, rat anti-CD34, and mouse anti-CD45 (all from Pharmingen, San Diego, Calif.), rat anti-laminin (BIODESIGN, Saco, Me.), and rabbit anti-β-galactosidase (ICN Pharmaceuticals, INC., Costa Mesa, Calif.). The LacZ gene was inserted in-frame into the original knock-out construct (Wigle et al. 1999) so that its product, β-gal, could be used in heterozygous and nullizygous Prox1 embryos to detect cells that in wild-type embryos, express Prox1. Primary antibodies were diluted in a solution of 20% heat-inactivated fetal bovine serum (HyClone Laboratories, Inc., Logan, Utah) and 2% blocking reagent (Roche, Indianapolis, Ind.) in maleate buffer (100 mM maleic acid, 150 mM NaCl; pH 7.5); overnight incubations were carried out in a humidified chamber at room temperature. The following Cy3-conjugated antibodies were used for fluorescence labeling: goat anti-rabbit IgG, donkey anti-goat IgG, and donkey anti-rat IgG (all from Jackson ImmunoResearch Laboratiories, Inc., West Grove, Pa.) and Alexa 488-conjugated goat anti-rabbit IgG (Molecular Probes, Eugene, Oreg.).

For immunohistochemical analyses, biotinylated donkey anti-rabbit IgG (Jackson ImmunoResearch Laboratories) and donkey anti-goat IgG (Santa Cruz Biotechnology, Santa Cruz, Calif.) were used in conjunction with the Vectastain ABC Kit (Vector Laboratories). The horseradish peroxidase-stained sections were counterstained with a 0.5% aqueous solution of methyl green (Sigma, St. Louis, Mo.).

Tumorigenesis Models: Two models of tumorigenesis were used. First, the xenograft experiment was performed as previously described (Streit, M. et. al. “Thrombospondin-2: a potent endogenous inhibitor of tumor growth and angiogenesis”, Proc Nat Acad Sci USA 96: 14888-93 (1999). Briefly, human A431 squamous carcinoma cells were injected (2×10⁶ cells per injection) intradermally into BALB/c (nu/nu) mice, and the mice were sacrificed 3 weeks later. Frozen tumor xenografts were cut into 6 μm sections and fixed for 30 minutes at 4° C. in 4% paraformaldehyde.

The second tumor model was a naturally occurring T-lymphoma that developed in the leg of a 6-month-old Ink4d nullizygous mouse. The tumor was dissected, fixed in 4% paraformaldehyde overnight at 4° C., cryoprotected in tissue-freezing medium, and cut into 10 μm sections on a cryostat. To discriminate between a lymphoma and a myogenic tumor, sections were stained either with anti-desmin or anti-CD45 antibodies. The tumor did not stain with anti-desmin antibodies but did stain with an anti-CD45 antibody.

1. A method for determining the extent of lymphatic involvement in a tumor comprising detecting the expression of Prox1 in said tumor.

2. The method of claim 1 wherein expression of said Prox1 is detected by making a measurement selected from the group consisting of:
a. measuring the amount of Prox1 mRNA; and
b. measuring the amount of Prox1 protein.

3. The method of claim 1 wherein expression of Prox1 is detected quantitatively in a sample taken from said tumor.

4. The method of claim 1 wherein expression of Prox1 in said tumor is detected with a marker that binds to Prox1 and can be visualized.

5. The method of claim 4 wherein said marker is fused to a Prox1 antibody.

6. A gene therapy vector comprising a gene encoding a Prox1 protein, wherein said vector is capable of expressing Prox1 protein in endothelial precursor cells.

7. A method for promoting the development of lymphatic tissue in a subject in need thereof comprising administering the gene therapy vector of claim 6.

8. A method of purifying endothelial precursor cells having the potential to develop into lymphatic tissue from a sample of cells comprising selecting cells from said sample which express a protein selected from the group consisting of Prox1 and LYVE-1.

9. The method of claim 8 wherein said sample of cells comprise cells that express CD31.

10. A method of purifying endothelial precursor cells having the potential to develop into lymphatic tissue from a sample of cells comprising selecting cells from said sample which express CD31 and a protein selected from the group consisting of Prox1 and LYVE-1.

11. A method for promoting the development of lymphatic tissue in a subject in need thereof comprising administering to said subject endothelial precursor cells purified according to the method of claim 8.
